(19) United States
(12) Patent Application Publication
(10) Pub. No.: US 2005/0171182 A1
(43) Pub. Date: Aug. 4, 2005

(54) Methods and compositions for use in the treatment of mutant receptor tyrosine kinase driven cellular proliferative diseases

(76) Inventor: Roger Briesewitz, Columbus, OH (US)

Correspondence Address: THERAVANCE, INC., 901 GATEWAY BOULEVARD, SOUTH SAN FRANCISCO, CA, 94080, US

(21) Appl. No.: 11/008,746

(22) Filed: Dec. 9, 2004

Related U.S. Application Data
(60) Provisional application No. 60/528,617, filed on Dec. 11, 2003.

(51) Int. Cl. A61K 31/407

(52) U.S. Cl. 514/410

(57) ABSTRACT

Methods for treating a subject suffering from a cellular proliferative disease characterized by the presence of a mutant receptor tyrosine kinase are provided. In practicing the subject methods, an effective amount of a CDK4 inhibitor, either alone or in combination with at least one of an inhibitor of the mutant receptor tyrosine kinase and an MEK inhibitor, is administered to the subject. Also provided are compositions, including pharmaceutical formulations and kits thereof, for practicing the subject methods.

### CROSS REFERENCE TO RELATED APPLICATIONS

This application claims the benefit of U.S. Provisional Application No. 60/528,617, filed on Dec. 11, 2003, the disclosure of which is incorporated herein by reference in its entirety.

### BACKGROUND OF THE INVENTION

1. Field of the Invention

This invention is directed to methods of treating cellular proliferative disorders characterized by the presence of a mutant receptor tyrosine kinase. This invention is also directed to compositions, kits, and systems, useful in such methods of treatment.

2. State of the Art

An accumulation of genetic changes underlies the development and progression of hyperproliferative disorders, such as cancer, resulting in cells that differ from normal cells in their behavior, biochemistry, genetics, and microscopic appearance. Mutations in DNA that cause changes in the expression level of key proteins, or in the structures and biological activities of proteins, are thought to be at the heart of cancer. For example, cancer can be triggered when genes that play a critical role in the regulation of cell growth and survival are mutated or overexpressed. Such “oncogenes” are involved in the dysregulation of growth that occurs in cancers.

Kinases are enzymes involved in phosphorylation that help regulate many cellular activities, particularly signaling from the cell membrane to the nucleus to initiate the cell's entrance into the cell cycle and to control other functions. For example, phosphorylation is important in signal transduction mediated by receptors via extracellular biological signals such as growth factors or hormones. Many oncogenes are kinases, i.e., enzymes that catalyze protein phosphorylation reactions.

In recent years, tyrosine kinases have emerged as promising drug targets in cancer, especially, kinases that are constitutively active because of an activating mutation. A prominent mutationally activated tyrosine kinase is bcr-abl, a fusion kinase that results from a reciprocal translocation between chromosomes 9 and 22. The resulting fusion gene called bcr-abl is sufficient to initiate chronic myeloid leukemia (CML). Other tyrosine kinases like c-Kit as well as PDGFRα and β are mutationally activated in a number of cancers and the mutations of these kinases have been linked to oncogenic transformation. Small activating deletions or point mutations of c-Kit are found in gastrointestinal stromal tumors (GIST) and systemic mast cell disease (SMCD). Like c-Kit, PDGFRα is found to be mutationally activated in GIST and also in hypereosinophilic syndrome (HES). In chronic myelomonocytic leukemia (CMML), PDGFRβ is expressed as a fusion kinase, Tel-PDGFRβ, as the result of a chromosomal translocation.

There is a continued need in the field for the development of new protocols for treating subjects suffering from cellular proliferative diseases.

Relevant Literature

U.S. Pat. No. 5,521,184; Baxter et al., Hum. Mol. Genet. 2002, 11, 1391-1397; Schaller et al., Med. Gen. Med. 2001, 3:9; and Lancet, 2002, 359, 1577-8.

Descriptions of CDK4 inhibitors can be found in: (1) U.S. Pat. Nos. 4,900,727, 5,733,920, 5,849,733, 6,040,321, 6,150,359, 6,262,096 B1, 6,498,163 B1, 6,569,878 B1, 6,593,326 B1, 6,630,464 B1, 6,720,332 B2, and 6,756,374 B2; (2) published U.S. Patent Application Nos. 2002/0151554A1, 2003/0149001, 2003/0087923, 2003/0203907, 2003/0229026, and 2004/0048915; (3) published PCT application nos. WO 98/49146, WO 00/12485, WO 01/14375, WO 01/44147, WO 02/20524, and WO 02/28861; and (4) published European Patent Application Nos. EP 1199306A1 and EP 1295878A1; as well as Webster, “The Therapeutic Potential of Targeting the Cell Cycle,” Exp. Opin. Invest. Drugs 1998, 7, 865-887 and Toogood, “Progress Toward The Development Of Agents To Modulate The Cell Cycle,” Curr. Opin. Cell. Biol. 2002, 6, 472-478.

Descriptions of MEK inhibitors can be found in: (1) U.S. Pat. Nos. 5,525,625, 6,251,943, 6,310,060, 6,638,945, 6,440,966, 6,455,582, 6,496,004, 6,506,798, 6,638,945 B1, 6,770,778 B2, and 6,809,106 B1; (2) published U.S. Patent Application Nos. 2003/0078428, 2003/0125359, 2003/0216460, 2003/0225076, 2004/0232869, 2004/0054172, and 2004/0006245; (3) published PCT application nos. WO 00/68201; WO 00/68200; WO 00/68199; WO 01/68619, WO 02/076496; WO 03/047585; WO 03/053960, WO 03/062189, WO 03/062191, and WO 03/077914; and (4) published Great Britain application No. GB 2323845; as well as Kolch, “Ras/Raf Signalling and Emerging Pharmacotherapeutic Targets,” Expert Opin. Pharmacother. 2002, 3, 709-718; Dancey, Current Pharmaceutical Design, 2002, 8, 2259-2267; Cox and Der, Cancer Biology & Therapy 2002, 1 599-606; Favata et al., J Biol. Chem. 1998, 272, 18623-18632; Zhao et al., J Antibiotics 1999, 52, 1086-1094; Williams et al., Biochemistry 1998, 37:9579-9585; Zhang et al., Bioorganic & Medicinal Chemistry Letters, 2001, 11, 1407-1410; and Hilger et al. Onkologie, 2002, 25, 511-518. Additional U.S. Patents of interest include: 5,821,072; 6,242,196; and 6,316,462.

### SUMMARY OF THE INVENTION

The present invention provides a method for treating a subject suffering from a cellular proliferative disease characterized by the presence of a mutant receptor tyrosine kinase, the method comprising administering to the subject a therapeutically effective amount of a CDK4 inhibitor. The invention also provides a method for treating a subject suffering from such a cellular proliferative disease, the method comprising administering to the subject a therapeutically effective amount of a CDK4 inhibitor in combination with a therapeutically effective amount of at least one of an inhibitor of the mutant receptor tyrosine kinase and an MEK inhibitor.

The invention also provides compositions comprising a CDK4 inhibitor; at least one of a mutant receptor tyrosine kinase inhibitor and an MEK inhibitor; and a pharmaceutically-acceptable carrier. In another aspect, the invention provides a kit comprising a CDK4 inhibitor and instructions for using the CDK4 inhibitor for treating a subject suffering from a cellular proliferative disease characterized by the presence of a mutant receptor tyrosine kinase. Such kits may further comprise at least one of an inhibitor of the mutant receptor tyrosine kinase and an MEK inhibitor.

The invention further provides a use of a CDK4 inhibitor in the manufacture of a medicament for the treatment of a cellular proliferative disease characterized by the presence of a mutant receptor tyrosine kinase; and a use of a CDK4 inhibitor in the manufacture of a medicament for administration in combination with at least one of an inhibitor of a mutant receptor tyrosine kinase and an MEK inhibitor for the treatment of a cellular proliferative disease characterized by the presence of the mutant receptor tyrosine kinase.

### BRIEF DESCRIPTION OF THE DRAWINGS

FIGS. 1A, 1B, and 1C show gene expression profiles of MV4-11 cells treated with THRX-165724 at a concentration of 300 nM.

FIG. 2 displays viability (%) of EOL-1 and BV173 cells incubated with imatinib (1 μM) in the absence (plain bar), and presence (patterned bar) of cytokines GM-CSF and IL-3.

FIG. 3 displays viability (%) of MV4-11 and THP-1 cells incubated with THRX-165724(1 μM) in the absence (plain bar), and presence (patterned bar) of cytokines GM-CSF and IL-3.

FIG. 4 displays viability (%) of EOL-1, MV4-11, and THP-1 cells incubated with arcyriaflavin (1 μM) in the absence (plain bar), and presence (patterned bar) of cytokines GM-CSF and IL-3.

FIG. 5 displays viability (%) of EOL-1, MV4-11, and THP-1 cells incubated with U0126 (10 μM) in the absence (plain bar), and presence (patterned bar) of cytokines GM-CSF and IL-3.

FIG. 6 displays viability (%) of EOL-1, MV4-11, and THP-1 cells incubated with arcyriaflavin (1 μM) and U0126 (10 μM) in the absence (plain bar), and presence (patterned bar) of cytokines GM-CSF and IL-3.

### DETAILED DESCRIPTION OF THE INVENTION

Methods for treating a subject suffering from a cellular proliferative disease characterized by the presence of a mutant receptor tyrosine kinase are provided. In practicing the subject methods, a therapeutically effective amount of a CDK4 inhibitor, either alone, or in combination with a therapeutically effective amount of at least one of an inhibitor of the mutant receptor tyrosine kinase and an MEK inhibitor, is administered to the subject. When a CDK4 inhibitor is administered in combination with an additional agent, the agents may be administered sequentially or simultaneously in the same or separate formulations. Also provided are compositions, including pharmaceutical formulations and kits thereof, for practicing the subject methods.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, particular methods, devices and materials are now described.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

The term “treatment” means that at least an amelioration of the symptoms associated with the condition afflicting the host is achieved, where amelioration is used in a broad sense to refer to at least a reduction in the magnitude of a parameter, e.g., symptom, associated with the condition being treated. As such, treatment also includes situations where the pathological condition, or at least symptoms associated therewith, is completely inhibited, e.g., prevented from happening, or stopped, e.g., terminated, such that the host no longer suffers from the condition, or at least the symptoms that characterize the condition.

The term “therapeutically effective amount” means an amount sufficient to effect treatment when administered to a subject or patient in need of treatment. The therapeutically effective amount may vary depending on the subject and disease state being treated, the severity of the affliction, the manner of administration, and whether an agent is administered alone or in combination with one or more other agents. For a given agent, the therapeutically effective amount may be determined by one of ordinary skill in the art.

All publications mentioned herein are incorporated herein by reference for the purpose of describing and disclosing the elements that are described in the publications which might be used in connection with the presently described invention.

In further describing the subject invention, the methods are described first in greater detail, followed by a review of representative applications in which the subject methods find use, as well as representative compositions, e.g., pharmaceutical formulations, kits and systems, that find use in practicing the subject methods.

Methods

As summarized above, the subject invention provides methods of treating a subject suffering from a cellular proliferative disease. The target cellular proliferative diseases that are the object of the subject methods are ones that are characterized by the presence of a mutant receptor tyrosine kinase. As a result of a mutation of a gene, such as a point mutation or a chromosomal translocation, such mutant receptor tyrosine kinases are constitutively active. As such, the mutant receptor tyrosine kinase is detectable in subjects that are treated according to the present invention. In certain embodiments, the disease afflicting the subject being treated according to the subject invention can be viewed as a disease that is caused, at least in part, by the activity of a mutant receptor tyrosine kinase.

The mutant receptor tyrosine kinases that characterize the cellular proliferative diseases whose treatment is the object of the subject methods are, in many embodiments, mutant receptor tyrosine kinases that confer an immortalized, and often hyperproliferative, phenotype onto a cell in which they are present. In other words, cells that express the subject mutant tyrosine kinases are ones that have an immortalized, and often hyperproliferative, phenotype. By “immortalized” is meant that the cell is immortal as determined using the assay described in Lab. Invest., 2002, 82, 323-333. By “hyperproliferative” is meant that the cell divides at an above normal rate, as determined using the assay described in Cancer Cell, 2002, 1: 421-432. In many embodiments, the mutant receptor tyrosine kinases are the products of mutated genes such that they are constantly signaling i.e. their signaling is not subject to the normal regulation that controls the wild type receptor tyrosine kinases.

As the receptor tyrosine kinases are mutant tyrosine kinases, they differ from the wild type tyrosine kinase of which they are a mutant in some manner, where the difference results in conference of the immortal and apoptosis-resistant phenotype on the cell harboring the mutant tyrosine kinase. By “apoptosis-resistant” is meant that the cell is less sensitive to a stimulus that promotes programmed cell death (apoptosis).

The mutant tyrosine kinases may be substitution or deletion mutants where in certain embodiments the mutant tyrosine kinases are fusion proteins.

Where the mutant tyrosine kinases are fusion proteins, the fusion proteins are typically characterized by having a C-terminal tyrosine kinase domain which is fused, either directly or through a linking domain, to an N-terminal domain that is from a different protein, i.e., is not from the same protein as the protein from which the C-terminal tyrosine kinase is obtained. In certain embodiments, the fusion of the N-terminal domain to the C-terminal tyrosine kinase domain leads to or provides for the kinase domain being constitutively active, which constitutive activity confers upon the cell the immortal phenotype. Examples of such fusion proteins are described, for example, in Griffin et al., Proc. Natl. Acad. Sci, 2003, 100, 7830-7835, Cools et al., N. Engl. J Med., 2003, 348, 1201-1214, and in US 2004/0045044 (the publication of Serial No. 10/637,356), the latter of which is incorporated herein by reference.

In certain embodiments the mutant receptor tyrosine kinases fall into the group of receptor kinases that are members of the PDGFR superfamily of tyrosine kinases. Representative specific tyrosine kinases of the PDGFR superfamily include, but are not limited to: Flt3, PDGFRα, PDGFRβ, c-Kit and VEGFR-2. In certain embodiments, the mutant tyrosine kinase is a mutant of a chromosome 4 tyrosine kinase, where by “chromosome 4 tyrosine kinase” is meant a tyrosine kinase whose genomic coding sequence is located on the human chromosome 4. Representative specific chromosome 4 tyrosine kinases of interest include, but are not limited to: PDGFRα, c-Kit and VEGFR-2. In yet other embodiments, the mutant receptor tyrosine kinases are not members of the PDGFR superfamily of tyrosine kinases, where representative non-PDGFR superfamily tyrosine kinases of interest include, but are not limited to: FGFR1, FGFR3, Ret, ALK, and the like. Another example of a non-PDGFR superfamily tyrosine kinase of interest is EGFR.

CDK4 Inhibitors

CDK4 (cyclin dependent kinase 4) inhibitors of interest may, in the broadest sense, be any compound that is capable of inhibiting the activity of CDK4. As such, of interest are general CDK inhibitors which can inhibit the activity of two or more different CDKs, e.g., CDK1, CDK2, as well as CDK4. Also of interest are CDK4 selective inhibitors. It is now understood that CDK4 and CDK6 are closely related kinases with virtually indistinguishable biochemical properties. The amino acid and nucleic acid sequence coding for human CDK4 and CDK6 can be found at Genbank accession numbers NM₁₃ 000075 and NM₁₃ 001259, respectively. The Genbank accession number U37022 also refers to CDK4. As used herein, CDK4 inhibitor refers to a compound that can be demonstrated to inhibit the activity of CDK4 or of CDK6. A given inhibitor is considered to be selective for CDK4 if its determined inhibitory activity for CDK4 is at least 5-fold, at least 10-fold, or at least 25-fold more potent than its determined inhibitory activity for CDK that is other than CDK4 and CDK6, e.g., CDK1, CDK2, etc.

A number of assays are known in the art for determining CDK4 inhibitory activity of a compound, where representative such assays are described in U.S. Pat. Nos., 6,040,321; 6,569,878; etc., where representative in vitro assays that find use evaluate, in a time dependent manner, a given compound's ability to inhibit the ability of CDK4 to incorporate radiolabeled phosphate donor into a protein substrate.

Representative specific CDK4 inhibitors include, but are not limited to the following compounds. Of interest in certain embodiments is the naturally occurring indolocarbazole arcyriaflavin A (1) 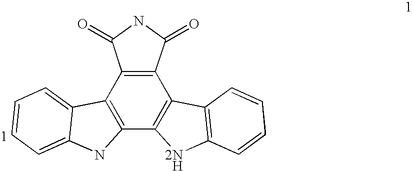
as well as substituted indolocarbazoles (see e.g., Zhu et al., J Med. Chem. 2003, 46, 2027-2030), such as a substituted indolocarbazole having fluoro and methyl substituents at the positions labeled 1 and 2, respectively, in 1 above. Other derivatives of interest include those described in U.S. Patent Application Publication Nos. 2003/0229026 and 2004/0048915 (or equivalently, WO 01/44247 and WO 02/28861, respectively) which disclose indolo[6,7-a]pyrrolo[3,4-c]carbazole-6,8-diones as potent CDK4 inhibitors.

Of interest in certain embodiments is the semi-synthetic flavopiridol (2) (also known as alvocidib), 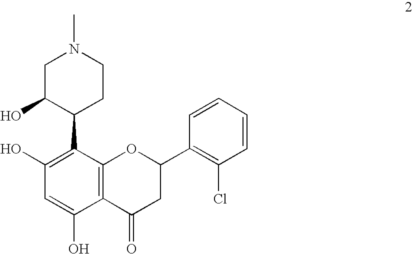
disclosed in U.S. Pat. No. 4,900,727; as well as analogs of flavopiridol, such as those reported in U.S. Pat. Nos. 5,733,920 and 5,849,733. However, in certain embodiments flavopiridol, or derivatives thereof, may not be employed.

Also of interest in certain embodiments are diarylurea derivatives, of which compound 3 is one example, 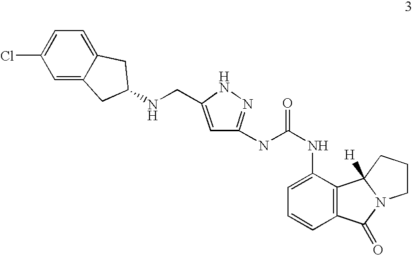
where such compounds are disclosed in EP 1199306 A1. In addition, patent publication US 2003/0203907 (or equivalently, EP 1295878 A1) describes structurally related 2(1H)-pyrazinone fused aromatic or heterocyclic derivatives having CDK4 and CDK6 activity, which compounds are also of interest.

Also of interest are diaminothiazoles, e.g., as reported in US 2002/0151554 (now U.S. Pat. No. 6,756,374 B2), where a specific example of such compounds is the compound denoted by the research code Ro-0506220 (4):

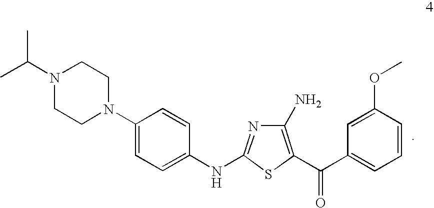

Also of interest in certain embodiments are napthyridinones of general structure 5, as disclosed in U.S. Pat. No. 6,150, 359, 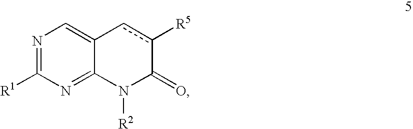
the pyrido[2,3-d]pyrimidines of general structure 6, as disclosed in U.S. Pat. No. 6,498,163 B1, 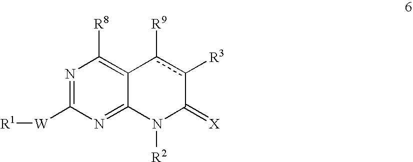
and the 2-(pyridin-2-ylamino)pyrido[2,3-d]pyrimidin-7-ones of general structure 7, as disclosed in patent publication US 2003/0149001 A1 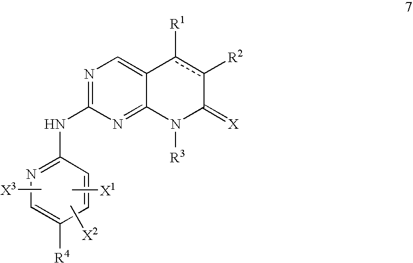
where the dashed line represents an optional bond and the substituents R¹, R², R³, R⁴, R⁵, R⁸, R⁹, X, X¹, X², X³, and W are defined in the respective publications. In particular, US 2003/0149001 discloses 6-acetyl-8-cyclopentyl-5-methyl-2-[5-(1-piperazinyl)pyridin-2-ylamino]pyrido[2,3-d]pyrimidin-7(8H)-one (7a) denoted by the research code PD-332991:

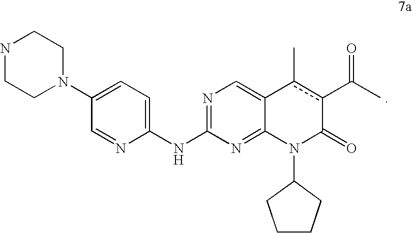

Further examples of compounds that inhibit CDK4 activity and are therefore of interest include, but are not limited to, the pyrimidine derivatives disclosed in WO 00/12485 and U.S. Pat. No. 6,593,326 B1, the imidazo[1,2-a]pyridine and pyrazolo[2,3-a]pyridine derivatives disclosed in WO 01/14375, the 4-amino-5-cyano-2-anilo-pyrimidine derivatives disclosed in the publication US 2003/0087923 A1, the aminothiazole compounds disclosed in U.S. Pat. Nos. 6,040,321, 6,262,096 B1, and 6,569,878 B1; the acridone and benzothiadiazine derivatives disclosed in U.S. Pat. No. 6,630,464 B1 (or equivalently WO 98/49146); and the oxindole derivatives disclosed in U.S. Pat. No. 6,720,332 B2 (or equivalently WO 02/20524). Yet further examples of CDK4 inhibitors are described in Toogood, Curr. Opin. Cell. Biol. 2002, 6, 472-478, Toogood, Med. Res. Rev., 2001, 21, 487-498, and in Carini et al., Bioorgan. Med. Chem. Lett., 2001, 11, 2209-2211.

Additional CDK4 inhibitory compounds may be readily identified by those of skill in the art using known CDK4 inhibitory assays, such as representative assays described above, where such identified compounds are also of interest for use in the subject methods.

As indicated above, in practicing the subject invention the amount of CDK4 inhibitor that is administered to the subject in need thereof is one that is effective to treat the condition afflicting the subject, as described in greater detail below, where the amount of CDK4 inhibitor that is employed in a given method may depend, at least in part, on whether the inhibitor is administered by itself, or in combination with one or more additional compounds.

Additional Inhibitors

In certain embodiments, the CDK4 inhibitor is administered to the subject in combination with one or more additional inhibitors of one or more additional protein activities, e.g., an inhibitor of the mutant receptor tyrosine kinase (i.e., a mutant receptor tyrosine kinase inhibitor); an MEK inhibitor; etc. By “in combination with” is meant that an amount of the CDK4 inhibitor agent is administered together with an amount of one or more additional inhibitors, such that two or more different inhibitors are administered to the subject, one of which is the CDK4 inhibitor. In certain embodiments, the inhibitor agents are administered sequentially, e.g., where the CDK4 inhibitor is administered before or after the other inhibitor(s). In yet other embodiments, the inhibitor agents are administered simultaneously, e.g., where the inhibitors are administered at the same time as two or more separate formulations or are combined into a single composition that is administered to the subject. Regardless of whether the inhibitor agents are administered sequentially or simultaneously, as illustrated above, the agents are considered to be administered together or in combination for purposes of the present invention. Routes of administration of the two agents may vary, where representative routes of administration are described in greater detail below.

Two specific representative inhibitors that may be administered in combination with the CDK4 inhibitor are: (1) inhibitors against mutant receptor tyrosine kinases; and (2) MEK inhibitors. As such, in certain embodiments, the CDK4 inhibitors are administered in combination with mutant tyrosine kinase inhibitors. In yet other embodiments, the CDK4 inhibitors are administered in combination with MEK inhibitors. In yet other embodiments, the CDK4 inhibitors are administered in combination with both mutant tyrosine kinase inhibitors and MEK inhibitors.

Mutant Receptor Tyrosine Kinase Inhibitors

Receptor tyrosine kinase inhibitors of interest may, in the broadest sense, be any compound that is capable of inhibiting the activity of the mutant receptor tyrosine kinase that characterizes the disease condition being treated. As such, of interest are general tyrosine kinase inhibitors which can inhibit the activity of two or more different tyrosine kinases, as well as selective inhibitors that demonstrate specific inhibitory activity primarily for the particular receptor tyrosine kinase that characterizes the disease condition. A variety of different types of agents may be employed as tyrosine kinase inhibitors, including but not limited to, e.g., small molecule agents, nucleic acid agents (e.g., antisense, RNAi), polypeptide agents, monoclonal antibodies etc.

In certain embodiments, the agents are pyrimidine derivatives as described in U.S. Pat. No. 5,521,184, the disclosure of which is herein incorporated by reference. In these embodiments, of interest are N-phenyl-2-pyrimidine-amine derivatives of formula (I): 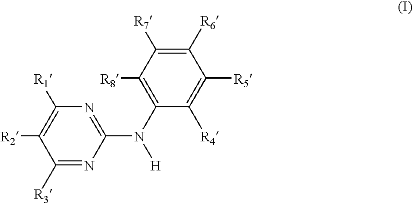
wherein

R_{1′} is 4-pyrazinyl, 1-methyl-1H-pyrrolyl, amino- or amino-lower alkyl-substituted phenyl wherein the amino group in each case is free, alkylated or acylated, 1H-indolyl or 1H-imidazolyl bonded at a five-membered ring carbon atom, or unsubstituted or lower alkyl-substituted pyridyl bonded at a ring carbon atom and unsubstituted or substituted at the nitrogen atom by oxygen,

R_{2′} and R_{3′} are each independently of the other hydrogen or lower alkyl,

one or two of the radicals R_{4′}, R_{5′}, R_{6′}, R_{7′} and R_{8′} are each nitro, fluoro-substituted lower alkoxy or a radical of formula (II):
—N(R_{9′})—C(═X)—(Y)ₖ—R₁₀ (II)
wherein

R_{9′} is hydrogen or lower alkyl,

X is oxo, thio, imino, N-lower alkyl-imino, hydroximino or O-lower alkyl-hydroximino,

Y is oxygen or the group NH,

k is 0 or 1 and

R₁₀ is an aliphatic radical having at least 5 carbon atoms, or an aromatic, aromatic-aliphatic, cycloaliphatic, cycloaliphatic-aliphatic, heterocyclic or heterocyclic-aliphatic radical,

and the remaining radicals R_{4′}, R_{5′}, R_{6′}, R_{7′} and R_{8′} are each independently of the others hydrogen, lower alkyl that is unsubstituted or substituted by free or alkylated amino, piperazinyl, piperidinyl, pyrrolidinyl or by morpholinyl, or lower alkanoyl, trifluoromethyl, free, etherified or esterifed hydroxy, free, alkylated or acylated amino or free or esterified carboxy,

and salts of such compounds having at least one salt-forming group.

In these embodiments:

1-Methyl-1H-pyrrolyl is preferably 1-methyl-1H-pyrrol-2-yl or 1-methyl-1H-pyrrol-3-yl.

Amino- or amino-lower alkyl-substituted phenyl R₁ wherein the amino group in each case is free, alkylated or acylated, is phenyl substituted in any desired position (ortho, meta or para) wherein an alkylated amino group is preferably mono- or di-lower alkylamino, for example dimethylamino, and the lower alkyl moiety of amino-lower alkyl is preferably linear C₁-C₃ alkyl, such as especially methyl or ethyl.

1H-Indolyl bonded at a carbon atom of the five-membered ring is 1H-indol-2-yl or 1H-indol-3-yl.

Unsubstituted or lower alkyl-substituted pyridyl bonded at a ring carbon atom is lower alkyl-substituted or preferably unsubstituted 2-, or preferably 3- or 4-pyridyl, for example 3-pyridyl, 2-methyl-3-pyridyl, 4-methyl-3-pyridyl or 4-pyridyl. Pyridyl substituted at the nitrogen atom by oxygen is a radical derived from pyridine N-oxide, i.e., N-oxido-pyridyl, e.g. N-oxido-4-pyridyl.

Fluoro-substituted lower alkoxy is lower alkoxy carrying at least one, but preferably several, fluoro substituents, especially trifluoromethoxy or preferably 1,1,2,2-tetrafluoro-ethoxy.

When X is oxo, thio, imino, N-lower alkyl-imino, hydroximino or O-lower alkyl-hydroximino, the group C═X is, in the above order, a radical C═O, C═S, C═N—H, C═N-lower alkyl, C═N—OH or CN—O-lower alkyl, respectively. X is preferably oxo.

k is preferably 0, i.e., the group Y is not present.

Y, if present, is preferably the group NH.

The term “lower” within the scope of this text denotes radicals having up to and including 7, preferably up to and including 4 carbon atoms.

Lower alkyl R_{1′}, R_{2′}, R_{3′} and R_{9′} is preferably methyl or ethyl.

An aliphatic radical R₁₀ having at least 5 carbon atoms preferably has not more than 22 carbon atoms, generally not more than 10 carbon atoms, and is such a substituted or preferably unsubstituted aliphatic hydrocarbon radical, that is to say such a substituted or preferably unsubstituted alkynyl, alkenyl or preferably alkyl radical, such as C₅-C₇ alkyl, for example n-pentyl. An aromatic radical R₁₀ has up to 20 carbon atoms and is unsubstituted or substituted, for example in each case unsubstituted or substituted naphthyl, such as especially 2-naphthyl, or preferably phenyl, the substituents preferably being selected from cyano, unsubstituted or hydroxy-, amino- or 4-methyl-piperazinyl-substituted lower alkyl, such as especially methyl, trifluoromethyl, free, etherified or esterified hydroxy, free, alkylated or acylated amino and free or esterified carboxy. In an aromatic-aliphatic radical R₁₀ the aromatic moiety is as defined above and the aliphatic moiety is preferably lower alkyl, such as especially C₁-C₂ alkyl, which is substituted or preferably unsubstituted, for example benzyl. A cycloaliphatic radical R₁₀ has especially up to 30, more especially up to 20, and most especially up to 10 carbon atoms, is mono- or poly-cyclic and is substituted or preferably unsubstituted, for example such a cycloalkyl radical, especially such a 5- or 6-membered cycloalkyl radical, such as preferably cyclohexyl. In a cycloaliphatic-aliphatic radical R₁₀ the cycloaliphatic moiety is as defined above and the aliphatic moiety is preferably lower alkyl, such as especially C₁-C₂ alkyl, which is substituted or preferably unsubstituted. A heterocyclic radical R₁₀ contains especially up to 20 carbon atoms and is preferably a saturated or unsaturated monocyclic radical having 5 or 6 ring members and 1-3 hetero atoms which are preferably selected from nitrogen, oxygen and sulfur, especially, for example, thienyl or 2-, 3- or 4-pyridyl, or a bi- or tri-cyclic radical wherein, for example, one or two benzene radicals are annellated (fused) to the mentioned monocyclic radical. In a heterocyclic-aliphatic radical R₁₀ the heterocyclic moiety is as defined above and the aliphatic moiety is preferably lower alkyl, such as especially C₁-C₂ alkyl, which is substituted or preferably unsubstituted.

Etherified hydroxy is preferably lower alkoxy. Esterified hydroxy is preferably hydroxy esterified by an organic carboxylic acid, such as a lower alkanoic acid, or a mineral acid, such as a hydrohalic acid, for example lower alkanoyloxy or especially halogen, such as iodine, bromine or especially fluorine or chlorine.

Alkylated amino is, for example, lower alkylamino, such as methylamino, or di-lower alkylamino, such as dimethylamino. Acylated amino is, for example, lower alkanoylamino or benzoylamino.

Esterified carboxy is, for example, lower alkoxycarbonyl, such as methoxycarbonyl.

A substituted phenyl radical may carry up to 5 substituents, such as fluorine, but especially in the case of relatively large substituents is generally substituted by only from 1 to 3 substituents. Examples of substituted phenyl that may be given special mention are 4-chloro-phenyl, pentafluoro-phenyl, 2-carboxy-phenyl, 2-methoxy-phenyl, 4-fluorophenyl, 4-cyano-phenyl and 4-methyl-phenyl.

Salt-forming groups in a compound of formula (I) are groups or radicals having basic or acidic properties. Compounds having at least one basic group or at least one basic radical, for example a free amino group, a pyrazinyl radical or a pyridyl radical, may form acid addition salts, for example with inorganic acids, such as hydrochloric acid, sulfuric acid or a phosphoric acid, or with suitable organic carboxylic or sulfonic acids, for example aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxybenzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethane-sulfonic acid, or aromatic sulfonic acids, for example benzene-, p-toluene- or naphthalene-2-sulfonic acid. When several basic groups are present mono- or poly-acid addition salts may be formed.

Compounds of formula (I) having acidic groups, for example a free carboxy group in the radical R₁₀, may form metal or ammonium salts, such as alkali metal or alkaline earth metal salts, for example sodium, potassium, magnesium or calcium salts, or ammonium salts with ammonia or suitable organic amines, such as tertiary monoamines, for example triethylamine or tri-(2-hydroxyethyl)-amine, or heterocyclic bases, for example N-ethylpiperidine or N,N′-dimethyl-piperazine.

Compounds of formula (I) having both acidic and basic groups can form internal salts.

Of particular interest in these embodiments is a pyrimidine derivative described in this patent in which R_{1′} is 3-pyridyl, R_{2′}, R_{3′}, R_{5′}, R_{6′}, and R_{8′} are each hydrogen, R_{4′} is methyl, and R_{7′} is a group of formula (II) in which R_{9′} is hydrogen, X is oxo, k is 0, and R₁₀ is 4-[(4-methyl-1-piperazinyl)methyl]phenyl. The mesylate salt of this compound having the chemical name 4-[(4-methyl-1-piperazinyl)methyl]-N-[4-methyl-3-[[4-(3-pyridinyl) -2-pyrimidinyl]amino-phenyl]benzamide methanesulfonate is now commonly known as imatinib mesylate and sold under the trademark Gleevec®.

In yet other embodiments of interest, the agent is not imatinib mesylate.

Also of interest are phthalazine compounds of formula (III), 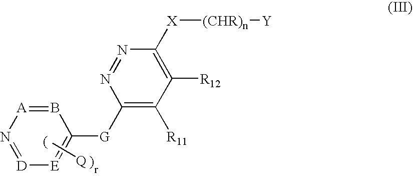
wherein r is 0 to 2, n is 0 to 2; m is 0 to 4; R₁₁ and R₁₂ (i) are in each case a lower alkyl, or (ii) together form a bridge in subformula (III*) 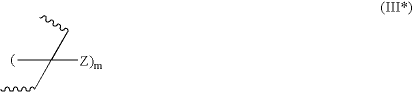
or (iii) together form a bridge in subformula (III**): 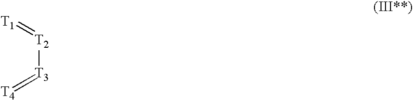
wherein one or two of the ring members T₁, T₂, T₃, and T₄ are nitrogen, and the remainder are in each case CH; A, B, D, and E are N or CH, wherein not more than 2 of these radicals are N; G is lower alkylene, acyloxy—or hydroxy-lower alkylene, —CH₂—O—, —CH₂—S—, —CH₂—NH—, oxa, thia, or imino; Q is lower alkyl, especially methyl; R is H or lower alkyl; X is imino, oxa, or thia; Y is aryl, pyridyl, or (un)substituted cycloalkyl; and Z is independently mono- or disubstituted amino, halogen, alkyl, substituted alkyl, hydroxy, etherified or esterified hydroxy, nitro, cyano, carboxy, esterified carboxy, alkanoyl, carbamoyl, N-mono- or N,N-disubstituted carbamoyl, amidino, guanidino, mercapto, sulfo, phenylthio, phenyl-lower alkylthio, alkylphenylthio, phenylsulfinyl, phenyl-lower alkylsulfinyl, alkylphenylsulfinyl, phenylsulfonyl, phenyl-lower alkylsulfonyl, or alkylphenylsulfonyl; and wherein the bonds characterized by a wavy line are either single or double bonds; or an N-oxide of said compound with the stipulation that, if Y is pyridyl or unsubstituted cycloalkyl, X is imino, and the remaining radicals are as defined, then G is selected from the group comprising lower alkylene, —CH₂—O—, —CH₂—S—, oxa and thia; or a salt thereof. Such compounds, e.g., PTK787 (also known as Vatalanib), are further described in the related documents WO 98/35958, U.S. patent application Ser. No. 09/859858 (now U.S. Pat. No. 6,514,974 B2), and U.S. Pat. No. 6,258,812 B1; the disclosure of the latter of which is herein incorporated by reference.

Also of interest in certain embodiments are the protein tyrosine kinase inhibitors of formula (IV): 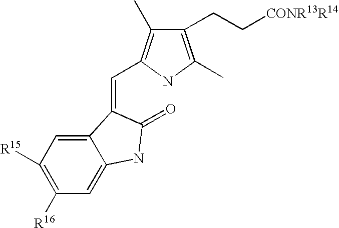
in which:

(i) R¹³ represents a hydrogen atom or a C₁₋₄alkyl group; and R¹⁴ represents a group of formula -A¹-NR¹⁷R¹⁸ in which each of R¹⁷ and R¹⁸ independently represents a hydrogen atom or a C₁₋₄alkyl group and A¹ represents (CH₂)_{m′}, (CH₂)_{n′}-A²-(CH₂)_{p′} or (CH₂CH₂O)_{q′}CH₂CH₂ in which m′ is an integer of from 2 to 10, each of n′ and p′ is an integer of from 1 to 6, A2 is CH═CH, phenylene, biphenylene, cyclohexylene or piperazinylene and q′ is 1, 2 or 3;

(ii) R¹³ and R¹⁴ together represent -A³-NR¹⁹-A⁴- in which each of A³ and A⁴ independently represents (CH₂)_{r′} or (CH₂CH₂O)_{s′}CH₂CH₂ in which r′ is an integer of from 2 to 6, s′ is 1, 2 or 3, and R¹⁹ represents a hydrogen atom or a C₁₋₄alkyl group;

(iii) R¹³ and R¹⁴ together with the nitrogen atom to which they are attached represent a piperidinyl group, which piperidinyl group bears a substituent of formula -A⁵-R²⁰ at the 4 position, in which A⁵ represents C₁₋₄alkylene and R²⁰ represents piperidin-4-yl; or

(iv) R¹³ and R¹⁴ together with the nitrogen atom to which they are attached represent a pyrrolidinyl, piperidinyl or morpholino group; and

R¹⁵ and R¹⁶ each independently represents a hydrogen atom, a halogen atom, a C₁₋₄alkyl group, a C₁₋₄alkoxy group, a phenyl group which is unsubstituted or substituted by one or two substituents selected independently from a halogen atom, a C₁₋₄alkyl group and a C₁₋₄alkoxy group, a group of formula R²¹S(O)₂NR²²—, a group of formula R²³N(R²⁴)S(O)₂—, a group of formula R²⁵C(O)N(R²⁶)— or a group of formula R²⁷N(R²⁸)C(O)— in which each of R²¹, R²³, R²⁵ and R²⁷ independently represents a C₁₋₄alkyl group or a phenyl group which is unsubstituted or substituted by one or two substituents selected independently from a halogen atom, a C₁₋₄alkyl group and a C₁₋₄alkoxy group, and each of R²², R²⁴, R²⁶ and R²⁸ independently represents a hydrogen atom or a C₁₋₄alkyl group;

or a pharmaceutically-acceptable salt thereof.

An inhibitor of formula (IV) of particular interest, identified as THRX-165724, is one in which R¹³ and R¹⁴ and the nitrogen to which they are attached form a piperazinyl ring and R¹⁵ and R¹⁶ are both hydrogen. Compounds of formula (IV) are described in U.S. patent application Ser. Nos. 60/343,746, 60/343,813, and in U.S. patent publication 2003/0171378 A1 (now U.S. Pat. No. 6,686,362 B2), the disclosures of which are herein incorporated by reference.

Another group of compounds of interest in certain embodiments are compounds of formula (V): 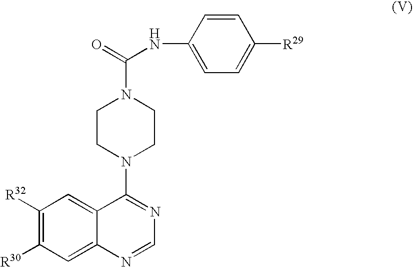
wherein:

R²⁹ is selected from the group consisting of —CN, —X, —CX₃, —R³³, —CO₂R³³, —SO₂R³³, —O—C₁₋₈alkyl that is straight or branched chained, —O-phenyl, —O-napthyl, —O-indolyl, and —O-isoquinolinyl, in which X is a halogen, and R³³ is hydrogen or a C₁₋₈alkyl that is straight or branched chained,

R³⁰ and R³² are each independently selected from the group consisting of —O—CH₃, —O—CH₂—CH₃, —O—CH₂—CH═CH₂, —O—CH₂—C≡CH, —O(CH₂)—SO₂—R³³, —O—CH₂—CH(R³⁴)CH₂—R³¹ and —O(—CH₂)_{n″}—R³¹, in which R³⁴ is —OH, —X, or a C₁₋₈alkyl that is straight or branched chained, n″ is 2 or 3, and

R³¹ is selected from the group consisting of; —OH, —O—CH₃, —O—CH₂—CH₃, —NH₂, —N(—CH₃)₂, —H—CH₂-phenyl, —NH-phenyl, —CN, —C(═NH)—NH₂, —NH—C(═NH)—NH₂, thiazolyl, oxazolyl, pyrrolidinyl, 4,4-difluoropiperidinyl, 3,3,-difluoropiperidinyl, 3,3-difluoropyrrolidinyl, morpholinyl, piperidinyl, imidazolyl, 1,2,3,-triazolyl, methylpiperidinyl, thiomorpholinyl 1,1-dioxide-thiomorpholinyl, —O-4-pyridinyl, 1H-tetrazolyl, piperazinyl, and 4-methylpiperazinyl; and pharmaceutically-acceptable isomers, salts, hydrates, solvates, and prodrug derivatives thereof.

Among compounds of formula (V), of particular interest is the compound, identified as MLN518 in which R²⁹ is —O-isopropyl, R³⁰ is —O—(CH₂)₃-4-morpholinyl, and R³² is —OCH₃. Compounds of formula (V) are further described in WO 02/16351, which is incorporated herein by reference.

Yet another group of compounds of interest in certain embodiments is compounds of formula (VI): 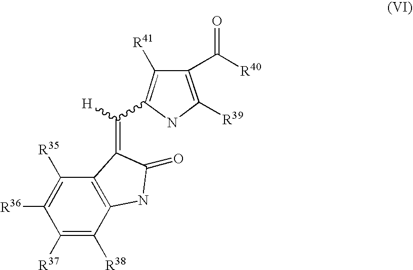
wherein:

R³⁵ is selected from the group consisting of hydrogen, halo, alkyl, cycloalkyl, aryl, heteroaryl, heteroalicyclic, hydroxy, alkoxy, —C(O)R⁴⁸, —NR⁴⁶R⁴⁷, —(CH₂)_{r*}R⁴⁹ and —C(O)NR⁴²R⁴³;

R36 is selected from the group consisting of hydrogen, halo, alkyl, trihalomethyl, hydroxy, alkoxy, cyano, , —NR⁴⁶R⁴⁷, —NR⁴⁶C(O)R⁴⁷, —C(O)R⁴⁸, aryl, heteroaryl, and —S(O)₂NR⁴⁶R⁴⁷;

R³⁷ is selected from the group consisting of hydrogen, halo, alkyl, trihalomethyl, hydroxy, alkoxy, —C(O)R⁴⁸, —NR⁴⁶R⁴⁷, aryl, heteroaryl, —NR⁴⁶S(O)₂R⁴⁷, —S(O)₂NR⁴⁶R⁴⁷, —NR⁴⁶C(O)R⁴⁷, —NR⁴⁶C(O)OR⁴⁷, and —S(O)₂R⁵³, wherein R⁵³ is alkyl, aryl, aralkyl, heteroaryl or heteroaralkyl;

R³⁸ is selected from the group consisting of hydrogen, halo, alkyl, hydroxy, alkoxy, and —NR⁴⁶R⁴⁷;

R³⁹ is selected from the group consisting of hydrogen, alkyl and —C(O)R⁴⁰;

R⁴¹ is selected from the group consisting of hydrogen, alkyl, aryl, heteroaryl, —C(O)R⁵⁰ and —C(O)R⁴⁰;

R⁴² and R⁴³ are independently selected from the group consisting of hydrogen, alkyl and aryl;

R⁴⁰ is selected from the group consisting of hydroxy, alkoxy, aryloxy, —(R⁴⁴)(CH₂)_{n*}R⁴⁵ and —NR46R⁴⁷;

R⁴⁴ is selected from the group consisting of hydrogen and alkyl;

R⁴⁵ is selected from the group consisting of —R⁴⁶R⁴⁷, hydroxy, —C(O)R⁴⁸, aryl, heteroaryl, —N⁺(O⁻)R⁴⁶R⁴⁷, —N(OH)R⁴⁶, and —NHC(O)R^{a}, wherein R^{a} is unsubstituted alkyl, haloalkyl, or aralkyl;

R⁴⁶ and R⁴⁷ are independently selected from the group consisting of hydrogen, alkyl, lower alkyl substituted with hydroxyalkylamino, cyanoalkyl, cycloalkyl, aryl, and heteroaryl; or

R⁴⁶ and R⁴⁷ may combine to form a heterocyclo group;

R⁴⁸ is selected from the group consisting of hydrogen, hydroxy, alkoxy, and aryloxy;

R⁴⁹ is selected from the group consisting of hydroxy, —C(O)R⁴⁸, —NR⁴⁶R⁴⁷ and —C(O)NR⁴⁶R⁴⁷;

R⁵⁰ is selected from the group consisting of alkyl, cycloalkyl, aryl and heteroaryl; and

n* and r* are independently 1, 2, 3, or 4;

or a pharmaceutically-acceptable salt thereof.

A compound of formula (VI) of particular interest, identified as SU11248, is the compound in which R³⁶ is fluoro, R³⁵, R³⁷, and R³⁸ are each hydrogen, R³⁹ and R⁴¹ are each methyl, and R⁴⁰ is —N(H)(CH₂)₂N(C₂H₅)₂. Compounds of formula (VI) are described in U.S. Pat. No. 6,573,293 B2 (or, equivalently WO 01/60814), the disclosure of which are incorporated herein by reference.

Also of interest are other protein tyrosine kinase inhibitors. Such inhibitors include, but are not limited to, the staurosporine derivatives, including the compound denoted by the research code PKC-412 or the generic name midostaurine, which are disclosed in WO 03/037347 to be useful for the treatment of diseases involving deregulated Flt3 receptor tyrosine kinase activity, and the tyrosine kinase inhibitors appearing in Appendix A of the United States provisional applications having Ser. Nos. 60/402,330 filed on Aug. 9, 2002 and 60/440,491 filed on Jan. 16, 2003; the disclosures of which are herein incorporated by reference.

MEK Inhibitors

MEK inhibitors of interest may, in the broadest sense, be any compound that is capable of inhibiting the activity of MEK. Two MEK protein kinase isoforms, encoded by two different genes, have been identified: MEK1 (also designated as MAPK/ERK kinase 1; protein kinase, mitogen-activated, kinase 1; PRKMK1 MKK1; or MAPKK1) and MEK2 (also designated as MAPK/ERK kinase2; protein kinase, mitogen-activated, kinase 2; PRKMK2 MKK2; or MAPKK2). The amino acid sequence and the nucleic acid sequence coding for the human MEK1 and MEK2 can be found at Genbank accession nos. L05624 and L11285, respectively. As used herein, MEK inhibitor refers to a compound that can be demonstrated to inhibit the activity of MEK1 or MEK2.

One representative MEK inhibitor is 2-(2-amino-3-methoxyphenyl)-4-oxo-4H-[1]benzopyran (8), 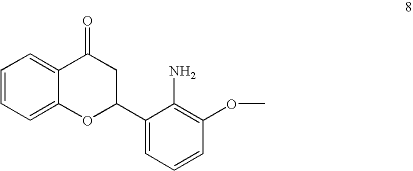
which is denoted by the research code PD-098059, and described in U.S. Pat. No. 5,525,625.

Another MEK inhibitor of interest is U-0126, (1,4-diamino-2,3-dicyano-1,4-bis[2-aminophenylthio]butadiene (9) 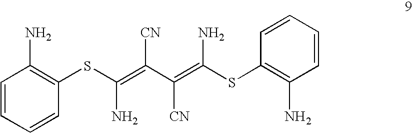
as described in Favata et al., J. Biol. Chem. 1998, 273, 18623-18632.

Also of interest as MEK inhibitors are naturally occurring resorcylic acid lactones, exemplified by L-783277 (10) (Zhao et al., J. of Antibiotics, 1999, 52, 1086-1094 and GB 2323845) and Ro-09-2210 (11) (Williams et al., Biochemistry, 1998, 37, 9579-9585)

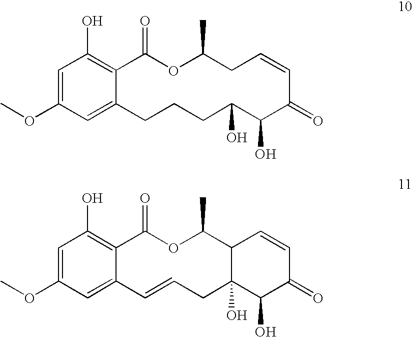

Also of interest as MEK inhibitors are 4-anilino-3-cyano-6,7-dialkoxyquinolinones, of which compound (12) 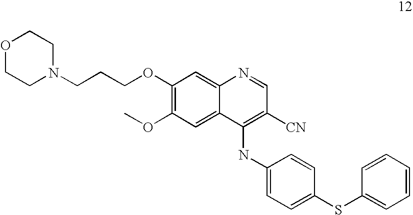
is a potent example, as been reported by Zhang et al., Bioorg. Med. Chem. Lett., 2001, 11, 1407-1410.

Also of interest as MEK inhibitors are bromo- or iodo phenylamino benzyhydroxamic acid derivatives, as disclosed in published United States patent publication US 2003/0078428, such as compound 13, denoted PD-184352 or CI-1040:

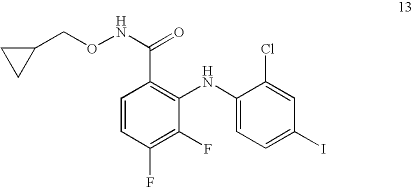

Additional, structurally-related examples of MEK inhibitors of interest which have a diarylamine core as in 13, include the phenylamino benzoic acid, benzamides, and benzyl alcohol derivatives disclosed in U.S. Pat. Nos. 6,251,943 B 1 and 6,310,060 B 1; the benzensulfonamide derivatives disclosed in U.S. Pat. No. 6,440,966 B 1; the sulfohydroxamic acid diarlyamine derivatives disclosed in U.S. Pat. No. 6,455,582 B1; the 4-arylamino, 4-aryloxy, and 4-arylthio diarylamine derivatives disclosed in U.S. Pat. No. 6,506,798 B1; the 5-amide substituted derivatives disclosed in US 2003/0225076 A1 (or equivalently WO 01/68619); and the N-4-substituted phenyl-anthranilic hydroxamate ester derivatives disclosed in U.S. Pat. No. 6,770,778 B2 and US 2004/0006245 A1 (or equivalently WO 03/062189 and WO 03/062191, respectively). Other examples include the N3 alkylated benzimidazole derivatives described in US 2003/0216460 A1 and US 2003/0232869 A1 (or equivalently WO 03/077914). Yet another related example is the compound denoted PD-325901 disclosed in US 2004/0054172 A1.

Other MEK inhibitor examples include the benzoheterocycle derivatives disclosed in U.S. Pat. No. 6,469,004 B1. Further examples of compounds that have been found to be MEK inhibitors include, but are not limited to, the quinolinone derivatives of general structural formulas 14, 15, and 16, which are disclosed in WO 00/68201, U.S. Pat. No. 6,809,106 B1 (or equivalently WO 00/68200), and U.S. Pat. No. 6,638,945 B1 (or equivalently WO 00/68199), respectively, 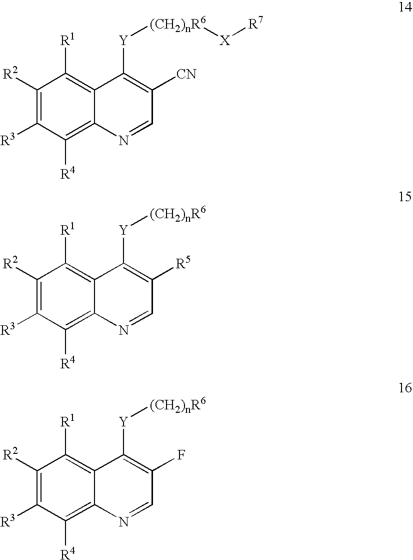
and the benzofuranyl substituted 3-cyanoquinoline derivatives of general formula 17, disclosed in WO 03/047585 and the related 3-cyanoquinoline derivatives of general formula 18, disclosed in WO 03/053960, 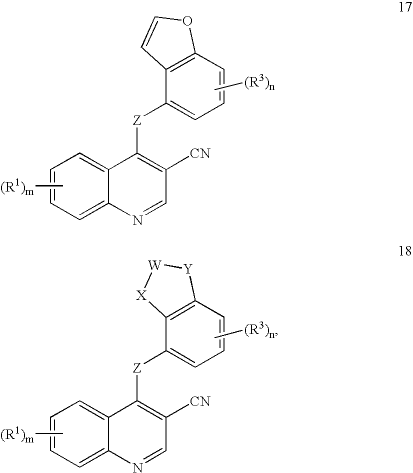
where all of the variables are defined in the respective publications. Yet other MEK inhibitors of interest include those described in Kolch, Expert Opin. Pharmacother., 2002, 3, 709-718.

Additional MEK inhibitory compounds may be readily identified by those of skill in the art using known MEK inhibitory assays. A number of assays are known in the art for determining MEK inhibitory activity of a compound, where representative such assays are described in U.S. Pat. Nos. 6,251,943; 6,310,060; 6,440,966; 6,455,582; 6,469,004; and 6,506,798; etc; the disclosures of which are herein incorporated by reference.

Dosages

The amounts of each agent that are administered to the subject in any given dosing may vary depending on the nature of the agent, the nature of condition being treated, the nature of the host being treated, and the like. Those of skill in the art will readily appreciate that dose levels can vary as a function of the specific compound, the nature of the delivery vehicle, and the like. Specific dosages for a given compound and treatment protocol are readily determinable by those of skill in the art by a variety of means.

The amount of the CDK4 inhibitor that is administered to the subject is an amount effective to treat the subject for the condition afflicting the subject, e.g., the cellular proliferative disease afflicting the subject, in view of the protocol being practiced. In certain representative embodiments, the amount of CDK4 inhibitor that is administered to the host ranges from about 0.01 to about 5000 mg per day.

The amount of the mutant receptor tyrosine kinase inhibitor, when employed, that is administered to the subject is an amount effective to treat the subject for the condition afflicting the subject, e.g., the cellular proliferative disease afflicting the subject, in view of the protocol being practiced. In certain representative embodiments, the amount of tyrosine kinase inhibitor that is administered to the host ranges from about 0.01 to about 5000 mg per day.

The amount of MEK inhibitor, when employed, that is administered to the subject is an amount effective to treat the subject for the condition afflicting the subject, e.g., the cellular proliferative disease afflicting the subject, in view of the protocol being practiced. In certain representative embodiments, the amount of MEK inhibitor that is administered to the host ranges from about 0.01 to about 5000 mg per day.

Routes of Administration

In practicing the subject methods, the active agents can be administered in a single daily dose or in multiple doses per day. The treatment regimen may require administration over extended periods of time, for example, for several days or for from one to six weeks, or over longer periods of time, including indefinitely. The amount per administered dose or the total amount administered will depend on such factors as the nature and severity of the infection, the age and general health of the patient, where representative amounts are provided above.

In the subject methods, the active agent(s) may be administered to the targeted cells using any convenient means capable of resulting in the desired modulation of fusion protein activity. Thus, the agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. As such, administration of the agents can be achieved in various ways, including oral, buccal, rectal, parenteral, intraperitoneal, intradermal, transdermal, intracheal, etc., administration.

A variety of hosts are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

Utility

The subject methods find use in the treatment of a variety of different conditions. As indicated above, by treatment is meant that at least an amelioration of the symptoms associated with the condition afflicting the host is achieved, where amelioration is used in a broad sense to refer to at least a reduction in the magnitude of a parameter, e.g., symptom, associated with the condition being treated. As such, treatment also includes situations where the pathological condition, or at least symptoms associated therewith, are completely inhibited, e.g., prevented from happening, or stopped, e.g., terminated, such that the host no longer suffers from the condition, or at least the symptoms that characterize the condition.

In many embodiments, the condition is a cellular proliferative disease condition characterized by the presence of a mutant receptor tyrosine kinase, as summarized above. There are many disorders associated with a dysregulation of cellular proliferation, i.e., cellular hyperproliferative disorders. The conditions of interest include, but are not limited to: leukemias, e.g., leukemias characterized by the presence of mutant receptor tyrosine kinases belonging to the PDGFR receptor family, such as AML (mutant Flt3), HES (mutant PDGFRα), systemic mast cell disease with eosinophilia (mutant c-Kit and mutant PDGFRα), chronic myelomonocytic leukemia (CMML) (mutant PDGFRβ), and the like; leukemias and myeloproliferative disorders characterized by the presence of mutant non-PDGFR receptor kinases, such as multiple myeloma (mutant FGFR3) and 8p11 myeloproliferative syndrome (mutant FGFR1), and the like; solid tumor cancers, e.g., those characterized by the presence of mutant PDGFR receptor kinases, such as gastrointestinal stromal tumor (mutant c-Kit, PDGFRα), etc.; those characterized by the presence of mutant non-PDGFR receptor kinases, such as bladder cancer (FGFR3) thyroid carcinoma (Ret) and anaplastic large cell lymphoma (ALK), and the like, as well as other types of cancers characterized by the presence of a mutant receptor tyrosine kinase.

Pharmaceutical Comnpositions

In one embodiment of the invention, pharmaceutical compositions comprising a CDK4 inhibitor; at least one of a mutant receptor tyrosine kinase inhibitor and an MEK inhibitor; and a pharmaceutically-acceptable carrier are provided. The active agents, e.g., in the form of a pharmaceutically acceptable salt, can be formulated for oral or parenteral administration for use in the subject methods, as described above. In these embodiments, a single formulation that includes all of the active agents (i.e., one composition that includes two or more active agents) is provided. In other embodiments, e.g., where the compounds are administered in combination as separate formulations, separate or distinct pharmaceutical compositions, each containing a different active agent, are provided.

By way of illustration, the active compound(s) can be admixed with conventional pharmaceutical carriers and excipients (i.e., vehicles) and used in the form of aqueous solutions, tablets, capsules, elixirs, suspensions, syrups, wafers, and the like. Such pharmaceutical compositions contain, in certain embodiments, from about 0.1 to about 90% by weight of the active compound, and more generally from about 1 to about 30% by weight of the active compound. The pharmaceutical compositions may contain common carriers and excipients, such as corn starch or gelatin, lactose, dextrose, sucrose, microcrystalline cellulose, kaolin, mannitol, dicalcium phosphate, sodium chloride, and alginic acid. Disintegrators commonly used in the formulations of this invention include croscarmellose, microcrystalline cellulose, corn starch, sodium starch glycolate and alginic acid.

A liquid composition will generally consist of a suspension or solution of the compound or pharmaceutically acceptable salt in a suitable liquid carrier(s), for example, ethanol, glycerine, sorbitol, non-aqueous solvent such as polyethylene glycol, oils or water, with a suspending agent, preservative, surfactant, wetting agent, flavoring or coloring agent. Alternatively, a liquid formulation can be prepared from a reconstitutable powder.

For example, a powder containing active compound, suspending agent, sucrose and a sweetener can be reconstituted with water to form a suspension; and a syrup can be prepared from a powder containing active ingredient, sucrose and a sweetener.

A composition in the form of a tablet can be prepared using any suitable pharmaceutical carrier(s) routinely used for preparing solid compositions. Examples of such carriers include magnesium stearate, starch, lactose, sucrose, microcrystalline cellulose and binders, for example, polyvinylpyrrolidone. The tablet can also be provided with a color film coating, or color included as part of the carrier(s). In addition, active compound can be formulated in a controlled release dosage form as a tablet comprising a hydrophilic or hydrophobic matrix.

A composition in the form of a capsule can be prepared using routine encapsulation procedures, for example, by incorporation of active compound and excipients into a hard gelatin capsule. Alternatively, a semi-solid matrix of active compound and high molecular weight polyethylene glycol can be prepared and filled into a hard gelatin capsule; or a solution of active compound in polyethylene glycol or a suspension in edible oil, for example, liquid paraffin or fractionated coconut oil can be prepared and filled into a soft gelatin capsule.

Tablet binders that can be included are acacia, methylcellulose, sodium carboxymethylcellulose, poly-vinylpyrrolidone (Povidone), hydroxypropyl methylcellulose, sucrose, starch and ethylcellulose. Lubricants that can be used include magnesium stearate or other metallic stearates, stearic acid, silicone fluid, talc, waxes, oils and colloidal silica.

Flavoring agents such as peppermint, oil of wintergreen, cherry flavoring or the like can also be used. Additionally, it may be desirable to add a coloring agent to make the dosage form more attractive in appearance or to help identify the product.

The compounds of the invention and their pharmaceutically acceptable salts that are active when given parenterally can be formulated for intramuscular, intrathecal, or intravenous administration.

A typical composition for intramuscular or intrathecal administration will be of a suspension or solution of active ingredient in an oil, for example, arachis oil or sesame oil. A typical composition for intravenous or intrathecal administration will be a sterile isotonic aqueous solution containing, for example, active ingredient and dextrose or sodium chloride, or a mixture of dextrose and sodium chloride. Other examples are lactated Ringer's injection, lactated Ringer's plus dextrose injection, Normosol-M and dextrose, Isolyte E, acylated Ringer's injection, and the like. Optionally, a co-solvent, for example, polyethylene glycol, a chelating agent, for example, ethylenediamine tetraacetic acid, and an anti-oxidant, for example, sodium metabisulphite may be included in the formulation. Alternatively, the solution can be freeze dried and then reconstituted with a suitable solvent just prior to administration.

The compounds of the invention and their pharmaceutically acceptable salts which are active on rectal administration can be formulated as suppositories. A typical suppository formulation will generally consist of active ingredient with a binding and/or lubricating agent such as a gelatin or cocoa butter or other low melting vegetable or synthetic wax or fat.

The compounds of this invention and their pharmaceutically acceptable salts which are active on topical administration can be formulated as transdermal compositions or transdermal delivery devices (“patches”). Such compositions include, for example, a backing, active compound reservoir, a control membrane, liner and contact adhesive. Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. No. 5,023,252, issued Jun. 11, 1991, herein incorporated by reference in its entirety. Such patches may be constructed for continuous, pulsatile, or on demand delivery of pharmaceutical agents.

Optionally, the pharmaceutical composition may contain other pharmaceutically acceptable components, such as buffers, surfactants, antioxidants, viscosity modifying agents, preservatives and the like. Each of these components is well-known in the art. See, for example, U.S. Pat. No. 5,985,310, the disclosure of which is herein incorporated by reference.

Other components suitable for use in the formulations of the present invention can be found in Remington's Pharmaceutical Sciences, Mace Publishing Company, Philadelphia, Pa., 17th ed. (1985).

Kits and Systems

Also provided are kits and systems that find use in practicing the subject methods, as described above. For example, kits and systems for practicing the subject methods may include one or more pharmaceutical formulations, which include at least a CDK4 inhibitor, and in certain embodiments one or more additional inhibitor compounds, in particular an MEK inhibitor, a mutant receptor tyrosine kinase inhibitor or both an MEK inhibitor and a mutant receptor tyrosine kinase inhibitor. As such, in certain embodiments the kits may include a single pharmaceutical composition, present as one or more unit dosages, where the composition may include one or more inhibitor compounds. In yet other embodiments, the kits may include two or more separate pharmaceutical compositions, each containing a different inhibitor compound.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The term “system” as employed herein refers to a collection of two or more different active agents, present in a single or disparate composition, that are brought together for the purpose of practicing the subject methods. For example, separately obtained CDK4 and MEK inhibitor dosage forms brought together and coadministered to a subject, according to the present invention, are a system according to the present invention.

The following examples are offered by way of illustration and not by way of limitation.

### EXPERIMENTAL

### Example I

### Demonstration that Mutant Flt3 Activates CyclinD/CDK4 Signaling in the MV4-11 Cell Line

The MV4-11 cell line is an acute myeloid leukemia cell line that expresses mutationally activated Flt3 (Leukemia, 2003, 17, 120-124). The mutant receptor tyrosine kinase inhibitor THRX-165724, discussed above, is a known Flt3 inhibitor as described in U.S. patent publication 2003/0171378 A1. The effect of THRX-165724 on gene expression in MV4-11 cells was evaluated as follows.

A ribonuclease protection assay (RPA) was performed according to the instructions provided by BD Biosciences Pharmingen (San Diego, Calif.). In this assay, a radiaoactively labeled probe of the gene or genes of interest was hybridized to target RNA in solution after which free probe and other single-stranded RNA were digested with RNAses. The reaction was resolved by polyacrylamide gel electrophoresis (PAGE) and the protected probe fragments were visualized as distinct bands by autoradiography. The higher the expression level of a gene of interest, the more radioactive probe was protected and the stronger the resulting band.

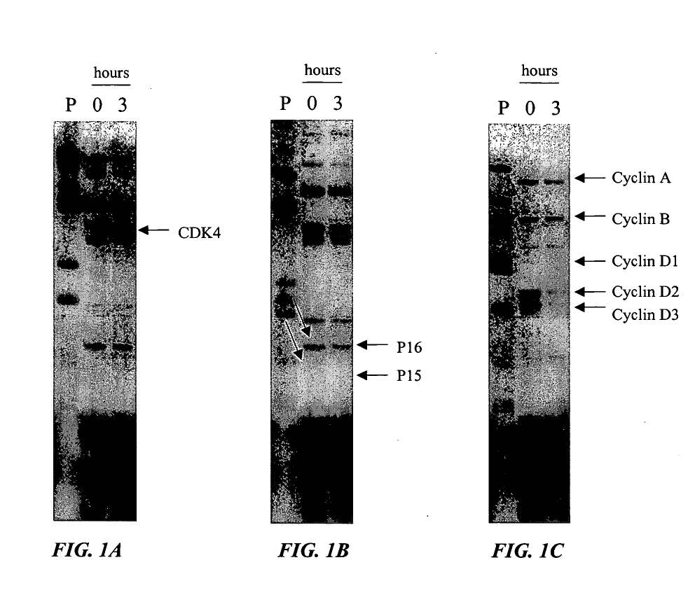

MV4-11 cells that had and had not been exposed to THRX-165724 (300 nM for 3 hour incubation) were evaluated by RPA, and the results are shown in FIGS. 1A-1C, where each band in the three lanes of each gel represents one gene. The lanes on the left of each gel, labeled “P”, contain the undigested probe, the lanes in the middle of each gel are the results for cells that were not exposed to the inhibitor, while those on the right are the results for cells that were exposed to THRX-165724 for 3 hours. Note that, after hybridization and RNAase digestion, the probe is reduced in size, which accounts for the shift between the location of the bands of interest in the lane corresponding to the undigested probe and in the other lanes. Results of the assay with a probe including a CDK4 specific probe are shown in FIG. 1A. Results of the assay for P 16 and P 15 expression (among other genes not labeled) are shown in FIG. 1B, while the expression of cyclin A, cyclin B, cyclin D1, cyclin D2 and cyclin D3 (as well as other genes not labeled) is shown in FIG. 1C.

The following may be observed from the RPA results:
Most genes are not affected in their expression level by treatment of the cells with THRX-165724, as evidenced by the similarity in banding patterns generated from the cells that were and were not exposed to THRX-165724 (i.e., lanes marked 0 and 3).
Three genes are affected and downregulated: cyclin D1, D2, D3, as shown in FIG. 1C where the bands for these three genes are much fainter in the cells exposed to THRX-165724.

It is known that cyclin D1, D2, D3 are positive regulators of CDK4 and that CDK4 is expressed in MV4-11 cells, as the protection of the CDK4 probe in FIG. 1A demonstrates. The intensity of the CDK4 band is the same in the sample derived from cells that were not treated and from cells that were treated with THRX-165724. This indicates that the expression of the CDK4 gene is not affected in response to treating MV4-11 cells with a Flt3 inhibitor. In addition, it is known that P15 and P 16 are negative regulators of cyclin D/CDK4 complexes (Cell Mol Life Sci., 2001, 58, 1907-1922. Leuk Lymphoma, 1996, 23, 505-520.) The RPA analysis also demonstrates that P16 is expressed in MV4-11 cells but P15 is not, as shown in FIG. 1B. It is also known that Cyclin D/CDK4 complexes regulate the transition of the cell cycle from G1 to S-phase. (Curr Opin Cell Biol., 2000, 12, :676-84).

The RPA experiment described above shows that in MV4-11 cells, mutated Flt3 upregulates the expression of cyclin D1, and especially cyclin D2 and D3. This is demonstrated by the drop in intensity of cyclin D1, D2 and D3 bands in the lane which represents the sample derived from MV4-11 cells treated with THRX-165724, the Flt3 inhibitor. During the period in which the cells were treated with the Flt3 inhibitor, the mutated Flt3 receptor is inactivated, so that any gene whose expression is driven by a signal that is derived from the mutant Flt3 is downregulated, which means fewer transcripts/ copies of mRNA for that particular gene are made in the cell. Since the loss of the Flt3 signal leads to the decrease of expression of cyclin D1, D2 and D3, the transcript levels of these genes drop. As a result, a smaller amount of probe is protected in the RPA experiment which is reflected in bands with reduced intensity. Since mutant Flt3 upregulates the expression of cyclin D1, D2 and D3, the mutant receptor indirectly also activates CDK4 since the activity of CDK4 is linked to the amount of cyclin D1, D2 and D3 present. Accordingly, mutant Flt3 activates cyclin D/CDK4 signaling.

### Example II

### Effect of a CDK4 inhibitor on the viability of cell lines characterized by a mutant receptor tyrosine kinase

Given that mutant Flt3 activates cyclin D/CDK4 signaling, the effect of the CDK4 inhibitor arcyriaflavin on the viability of MV4-11 cells was tested. As a control in this experiment, an inhibitor for cyclin A/CDK2 and cyclin E/CDK2 (purvalanol) as well as an inhibitor for cyclin B/CDK1 (alsterpaullone) was included. Additional control compounds in this experiment were the Flt3 inhibitor THRX-165724 and the PDGFRα inhibitor imatinib mesylate (Gleevec®, hereinafter “imatinib”). The inhibitors were also tested against EOL-1 cells, which express the mutant fusion kinase FiplL1-PDGFROα and against K562 and BV173 cells (CML cell lines) and THP-1 and U937 cells (AML cell lines). The dilution range of the inhibitors was from 20 μM to 10 nM.

Viability of the cell lines was determined by an MTT assay, which is based on the reduction of the tetrazolium salt 3, [4,5-dimethylthiazol-2-yl]-2,5-diphenyltetrazolium bromide (MTT). Mitochondrial enzymes associated with metabolic activity reduce MTT to a formazan dye, which can be measured spectrometrically. Cells undergoing apoptosis show reduced metabolic activity resulting in reduced formation of the formazan dye. Results are quantitated by comparison with an untreated control.

Viablity results expressed in terms of the quantity denoted IC₅₀, i.e. the concentration of inhibitor at which the viability of the cells is reduced by 50%, are displayed in Table 1.

TABLE 1
Viability (IC₅₀(μM)) of Cell Lines Exposed to Inhibitors for 72 Hours

| Inhibitor | Target | K562 | BV173 | EOL-1 | MV4-11 | THP-1 | U937 |
|---|---|---|---|---|---|---|---|
| arcyriaflavin | CDK4/cyclinD | >10 | >10 | 0.2 | 0.5 | >10 | >10 |
| purvalanol | CDK2/A, CDK2/E | >10 | >10 | >10 | 3 | >10 | >10 |
| alsterpaullone | CDK1/cyclin B | >10 | 1 | 1 | 1 | 1 | 0.6 |
| THRX-165724 | Flt3, PDGFRα | >10 | >10 | 0.02 | 0.05 | >10 | >10 |
| imatinib | Bcr-abl, PDGFRα | 0.3 | 0.6 | <.001 | >10 | >10 | >10 |

As can be seen from the above results, arcyriaflavin selectively reduces the viability of EOL-1 and MV4-11 cells. In contrast, cells that do not express a mutationally activated receptor tyrosine kinase are not sensitive to the inhibition of CDK4 by arcyriaflavin. K562 and BV173 are CML cell lines that express the bcr-abl fusion kinase. Like the AML cell lines THP-1 and U937, K562 and BV173 are not sensitive to arcyriaflavin. However, K562 and BV173 are sensitive to imatinib which is an inhibitor of bcr-abl. Although abl is a tyrosine kinase which is mutated in BV173 and K562, like mutant PDGFRa in EOL-1 and mutant Flt3 in MV4-11, mutant abl does not sensitize these cells to arcyriaflavin. Only cells with mutant receptor tyrosine kinases are sensitive to the inhibition of CDK4.

Since, as shown in Example I above, mutant Flt3 upregulates cyclin D/CDK4 activity in MV4-11 cells, the effect of the CDK4 inhibitor arcyriaflavin on the viability of these cells shows that the upregulation of cyclin D/CDK4 is a critical step in the survival and proliferation pathways activated by mutant Flt3.

The observation that EOL-1 cells, like MV4-11 cells, are sensitive to arcyriaflavin shows that the oncokinase Fip1L1-PDGFRα activates cyclin D/CDK4 as well. Since Flt3 and PDGFRα both belong to the PDGFR superfamily of receptor tyrosine kinases, these results demonstrate that CDK4 inhibitors selectively reduce the viability of cells characterized by the presence of mutant receptor tyrosine kinases.

### EXAMPLE III

### Effect of Inhibitors in the Presence of Cytokines

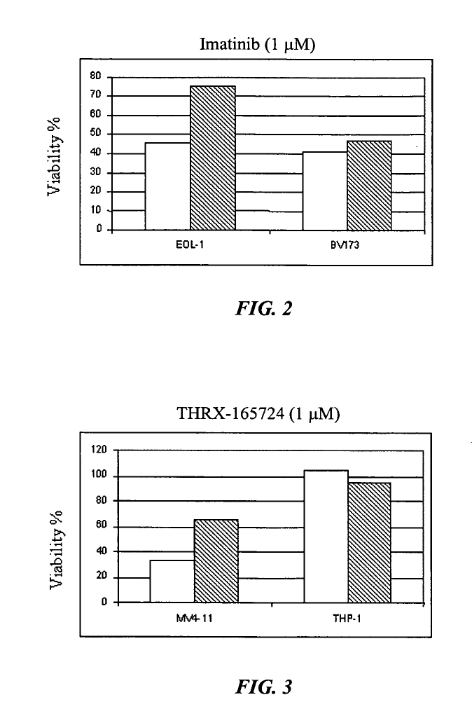

Recent clinical trials of Flt3 inhibitors for treatment of AML patients showed a less pronounced reduction of AML blasts in bone marrow than in peripheral blood in some patients. The bone marrow environment is known to contain a multitude of cytokines. The effect of cytokines on the activity of THRX-165724 and imatinib against MV4-11 and EOL-1 cells, respectively, was investigated. The cell lines were incubated with the appropriate inhibitor in the presence and absence of the cytokines GM-CSF and IL-3 (10 ng/mL each). The CML cell line BV173, which is sensitive to imatinib, as well as the THP-1 cell line, which is not sensitive to imatinib or THRX-165724, were included as controls. Results of the MTT viability assay for the EOL-1 and BV173 cell lines incubated with imatinib (1 μM) and for the MV4-11 and THP-1 cell lines incubated with THRX-165724 (1 μM) for 48 hours are shown in FIGS. 2 and 3, respectively.

It was found that THRX-165724 and imatinib strongly reduce the viability of MV4-11 and EOL-1 cells in the absence of cytokines; however, in the presence of cytokines the viability of the cells is much less affected.

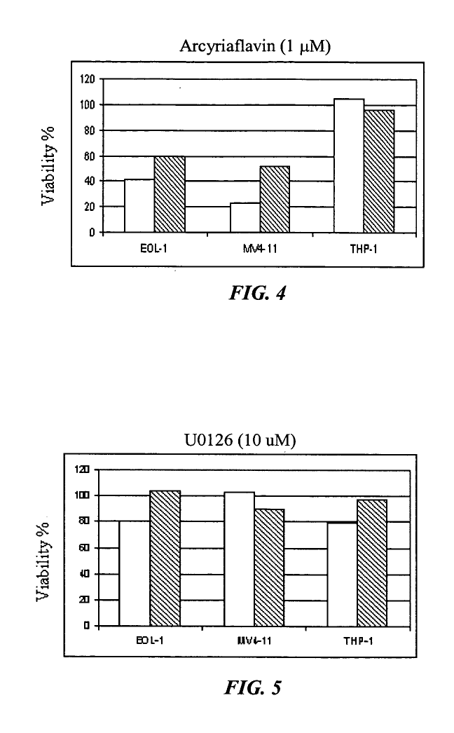

Like the specific inhibitors for mutationally activated tyrosine kinases, the CDK4 inhibitor arcyriaflavin can induce apoptosis in MV4-11 and EOL-1 cells (see Table 1). In order to study the effect of cytokines on the potency of arcyriaflavin, MV4-11 and EOL-1 cells were incubated with arcyriaflavin (1 μM) for 48 hours in the presence and absence of GM-CSF and IL-3 and viability, as determined by MTT assay, is shown in FIG. 4. In the presence of these cytokines the effect of arcyriaflavin on the viability of MV4-11 and EOL-1 cells is much reduced, similar to the reduction seen when MV4-11 and EOL-1 cells are exposed to THRX-165724 and imatinib in the presence of cytokines.

### Example IV

### Combination of a CDK4 Inhibitor and an MEK Inhibitor on the Viability of Cell Lines Characterized by Mutant Receptor Tyrosine Kinases

In hematopoietic cells, the cytokines GM-CSF and IL-3 have been shown to provide survival and proliferation signaling through the activation of MEK. To test the role of MEK in the survival of MV4-11 and EOL-1 cells, a viability assay (48 hour incubation) was performed using U0126 (10 μM), an MEK specific inhibitor (see FIG. 5). By itself, U0126 slightly alters the survival or proliferation of the cell lines tested.

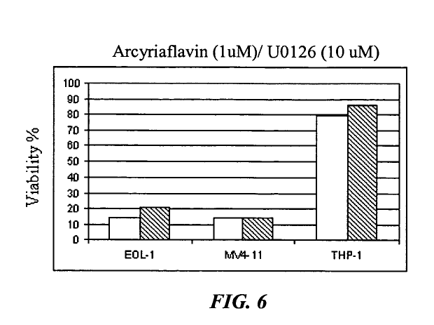

MV4-11 and EOL-1 cells were incubated with a combination of UO126 (10 μM), the MEK specific inhibitor, and arcyriaflavin (1 μM), the CDK4 inhibitor for 48 hours and results are shown in FIG. 6. As can be seen in the figure, the combination of an MEK specific inhibitor and a CDK4 inhibitor acts synergistically in reducing the viability of MV4-11 and EOL-1 cells and potently reduces viability even in the presence of cytokines. The control cell line, THP-1, which does not express a mutationally activated tyrosine kinase, are not affected by arcyriaflavin alone or in combination with U0126.

Results of an independent set of MTT viability assays in which EOL-1, MV4-11, and THP-1 cells were incubated with the listed agents in the absence and presence of the cytokines GM-CSF and IL-3 are summarized in Table 2.

TABLE 2
Viability (%) of Cell Lines Exposed to Inhibitors^{#} for 48 Hours

| | | GM-CSF/IL-3 |
|---|---|---|
| Inhibitor | Cytokines Absent | (10 ng/mL each) |
| EOL-1 cell line | | |
| imatinib | 46 | 76 |
| arcyriaflavin | 42 | 60 |
| U0126 | 80 | 103 |
| arcyriaflavin/U0126 | 14 | 21 |
| imatinib/U0126 | 12 | 44 |
| MV4-11 cell line | | |
| THRX-165724 | 33 | 65 |
| arcyriaflavin | 23 | 52 |
| U0126 | 103 | 90 |
| arcyriaflavin/U0126 | 14 | 14 |
| THRX-165724/U0126 | 23 | 55 |
| THP-1 cell line | | |
| THRX-165724 | 105 | 95 |
| arcyriaflavin | 105 | 96 |
| U0126 | 79 | 97 |
| arcyriaflavin/U0126 | 79 | 86 |
| THRX-165724/U0126 | 80 | 86 |

^{#}U0126 (10 μM), all other inhibitors at 1 μM

It is apparent, from Table 2, as well, that the combination of an MEK specific inhibitor (U0126) and a CDK4 inhibitor (arcyriaflavin) reduces the viability of MV4-11 and EOL-1 cells, i.e. the cell lines expressing mutant receptor tyrosine kinases, even in the presence of cytokines.

It is evident from the above results and discussion that the subject invention provides important new methods of treatment for cellular proliferative disease conditions, which will provide significant benefits to the medical and related fields. Accordingly, the subject invention represents a significant contribution to the art.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A method of treating a subject suffering from a cellular proliferative disease characterized by the presence of a mutant receptor tyrosine kinase, the method comprising:
administering to the subject a therapeutically effective amount of a CDK4 inhibitor.

2. The method according to claim 1, wherein the cellular proliferative disease is a leukemia.

3. The method according to claim 1, wherein the cellular proliferative disease is characterized by the presence of a solid tumor.

4. The method according to claim 1, wherein the mutant receptor tyrosine kinase is a member of the PDGFR tyrosine kinase superfamily.

5. The method according to claim 1, wherein the mutant receptor tyrosine kinase is selected from PDGFRα, PDGFRβ, Flt3, FGFR1, FGFR3, Ret, ALK, and EGFR.

6. The method according to claim 1, wherein the method further comprises administering to the subject a therapeutically effective amount of an inhibitor of the mutant receptor tyrosine kinase.

7. The method according to claim 1, wherein the method further comprises administering to the subject a therapeutically effective amount of an MEK inhibitor.

8. The method according to claim 1, wherein the method further comprises administering to the subject a therapeutically effective amount of an inhibitor of the mutant receptor tyrosine kinase and a therapeutically effective amount of an MEK inhibitor.

9. The method according to claim 1, wherein the method further comprises screening the subject to identify the presence of the mutant receptor tyrosine kinase.

10. The method according to claim 1, wherein the subject is a human.

11. The method according to claim 5, wherein the method further comprises administering to the subject a therapeutically effective amount of an inhibitor of the mutant receptor tyrosine kinase.

12. The method according to claim 5, wherein the method fuirther comprises administering to the subject a therapeutically effective amount of an MEK inhibitor.

13. The method according to claim 5, wherein the method further comprises administering to the subject a therapeutically effective amount of an inhibitor of the mutant receptor tyrosine kinase and a therapeutically effective amount of an MEK inhibitor.

14. The method according to claim 5, wherein the method further comprises screening the subject to identify the presence of the mutant receptor tyrosine kinase.

15. The method according to claim 5, wherein the subject is a human.

16. A composition comprising:
a) a CDK4 inhibitor;
b) at least one of:
i) a mutant receptor tyrosine kinase inhibitor; and
ii) an MEK inhibitor; and
c) a pharmaceutically-acceptable carrier.

17. The composition according to claim 16, wherein the composition comprises a mutant receptor tyrosine kinase inhibitor.

18. The composition according to claim 16, wherein the composition comprises an MEK inhibitor.

19. The composition according to claim 16, wherein the composition comprises a mutant receptor tyrosine kinase inhibitor and an MEK inhibitor.

20. A kit comprising:
a) a CDK4 inhibitor; and
b) instructions for using the CDK4 inhibitor to treat a subject suffering from a cellular proliferative disease characterized by the presence of a mutant receptor tyrosine kinase.

21. The kit according to claim 20, wherein the kit fuirther comprises at least one additional inhibitor selected from:
i) an inhibitor of the mutant receptor tyrosine kinase; and
ii) an MEK inhibitor

22. The kit according to claim 21, wherein the kit comprises an inhibitor of the mutant receptor tyrosine kinase.

23. The kit according to claim 21, wherein the kit comprises an MEK inhibitor.

24. The kit according to claim 21, wherein the kit comprises an inhibitor of the mutant receptor tyrosine kinase and an MEK inhibitor.

25. The kit according to claim 21, wherein the CDK4 inhibitor and the at least one additional inhibitor are present in a single formulation.

26. The kit according to claim 21, wherein the CDK4 inhibitor and the at least one additional inhibitor are present as separate formulations.

27. A system comprising:
a) a CDK4 inhibitor; and
b) at least one of:
i) a mutant receptor tyrosine kinase inhibitor; and
ii) an MEK inhibitor.

28. The system according to claim 27, wherein the system comprises a mutant receptor tyrosine kinase inhibitor.

29. The system according to claim 27, wherein the system comprises an MEK inhibitor.

30. The system according to claim 27, wherein the system comprises a mutant receptor tyrosine kinase inhibitor and an MEK inhibitor.
